(19) United States
(12) Patent Application Publication
(10) Pub. No.: US 2021/0225460 A1
(43) Pub. Date: Jul. 22, 2021

(54) EVALUATING THE ROBUSTNESS AND TRANSFERABILITY OF PREDICTIVE SIGNATURES ACROSS MOLECULAR BIOMARKER DATASETS

(71) Applicant: Genialis Inc., Houston, TX (US)

(72) Inventors: Miha Stajdohar, Ljubljana (SI); Luka Ausec, Kranj (SI); Matjaz Zganec, Ruse (SI); Rafael Rosengarten, San Ramon, CA (US)

(21) Appl. No.: 17/154,683

(22) Filed: Jan. 21, 2021

Related U.S. Application Data
(60) Provisional application No. 62/963,735, filed on Jan. 21, 2020.

(51) Int. Cl. G16B 50/30; G16B 40/00; G16H 50/20; C12Q 1/6883

(52) U.S. Cl. CPC G16B 50/30; G16B 40/00; C12Q 2600/156; C12Q 1/6883; C12Q 2600/112; G16H 50/20

(57) ABSTRACT

Embodiments of the present disclosure relate to analysis of gene and other molecular biomarker signatures, and more specifically, to evaluating the robustness and transferability of predictive signatures across genomic, proteomic, or metabolomic datasets.

### CROSS-REFERENCE TO RELATED APPLICATIONS

This application claims the benefit of U.S. Provisional Application No. 62/963,735, filed Jan. 21, 2020, which is hereby incorporated by reference in its entirety.

### BACKGROUND

Embodiments of the present disclosure relate to analysis of gene and other molecular biomarker signatures, and more specifically, to evaluating the robustness and transferability of predictive signatures across genomic, proteomic, or metabolomic datasets.

### BRIEF SUMMARY

According to embodiments of the present disclosure, methods of and computer program products for determining a transferable molecular biomarker signature is provided. In various embodiments, at least one signature is read. Each signature relates a first plurality of molecular biomarkers to one of a plurality of output classifications. For each of a plurality of datasets, an expression value of each of the first plurality of molecular biomarkers is normalized for each of the plurality of output classifications, yielding a plurality of normalized expressions, each associated with one of the first plurality of molecular biomarkers, one of the plurality of output classifications, and one of the plurality of datasets. For each of the first plurality of molecular biomarkers, a pairwise comparison is performed between the normalized expressions associated with that molecular biomarker. Each pairwise comparison is between normalized expressions associated with a same output classification and a different dataset, thereby determining a transferability score for each of the plurality of molecular biomarkers. The first plurality of molecular biomarkers is ranked based on its transferability score. A second plurality of molecular biomarkers is generated from the first plurality of molecular biomarkers by applying a transferability score threshold to the first plurality of molecular biomarkers.

In some embodiments, each of the first plurality of molecular biomarkers is a gene. In some embodiments, each of the first plurality of molecular biomarkers is a protein. In some embodiments, each signature comprises a mapping function. In some embodiments, each signature comprises a plurality of synaptic weights. In some embodiments, each output classification comprises a phenotype. In some embodiments, the phenotype is a disease phenotype. In some embodiments, said normalization comprises quantile normalization. In some embodiments, said normalization is to a predetermined reference distribution. In some embodiments, performing the pairwise comparison comprises computing a Kolmogorov-Smirnov statistic.

In some embodiments, determining the transferability score comprises computing a mean of the pairwise comparisons. In some embodiments, the plurality of datasets comprises at least one dataset derived from each of a plurality of platform technologies. In some embodiments, the platform technologies comprise microarrays and RNA-sequencing. In some embodiments, the platform technologies comprise mass spectrometry, ELISA, antibody arrays, peptide fingerprinting, and/or protein barcoding. In some embodiments, each of the plurality of datasets are derived from the same biological samples.

According to embodiments of the present disclosure, a computing node comprising a computer readable storage medium having program instructions embodied therewith is provided. The program instructions are executable by a processor of the computing node to cause the processor to perform a method as follows. A first signature is read. The first signature relates a first plurality of molecular biomarkers to a first of a plurality of output classifications. For each of a plurality of datasets, an expression value of each of the first plurality of molecular biomarkers is normalized for each of the plurality of output classifications, yielding a plurality of normalized expressions, each associated with one of the first plurality of molecular biomarkers, one of the plurality of output classifications, and one of the plurality of datasets. For each of the first plurality of molecular biomarkers, a pairwise comparison is performed between the normalized expressions associated with that molecular biomarker. Each pairwise comparison is between normalized expressions associated with a same output classification and a different dataset, thereby determining a transferability score for each of the plurality of molecular biomarkers. The first plurality of molecular biomarkers is ranked based on its transferability score. A second plurality of molecular biomarkers is generated from the first plurality of molecular biomarkers by applying a transferability score threshold to the first plurality of molecular biomarkers.

In some embodiments, each of the first plurality of molecular biomarkers is a gene. In some embodiments, each of the first plurality of molecular biomarkers is a protein. In some embodiments, each signature comprises a plurality of synaptic weights. In some embodiments, each signature comprises a mapping function. In some embodiments, each output classification comprises a phenotype. In some embodiments, the phenotype is a disease phenotype. In some embodiments, said normalization comprises quantile normalization. In some embodiments, said normalization is to a predetermined reference distribution. In some embodiments, performing the pairwise comparison comprises computing a Kolmogorov-Smirnov statistic.

In some embodiments, determining the transferability score comprises computing a mean of the pairwise comparisons. In some embodiments, the plurality of datasets comprises at least one dataset derived from each of a plurality of platform technologies. In some embodiments, the platform technologies comprise microarrays and RNA-sequencing. In some embodiments, the platform technologies comprise mass spectrometry, ELISA, antibody arrays, peptide fingerprinting, and/or protein barcoding. In some embodiments, each of the plurality of datasets are derived from the same biological samples.

In various embodiments, a computer readable storage medium having program instructions embodied therewith is provided, the program instructions executable by a processor to cause the processor to perform a method as follows. At least one signature is read. Each signature relates a first plurality of molecular biomarkers to one of a plurality of output classifications. For each of a plurality of datasets, an expression value of each of the first plurality of molecular biomarkers is normalized for each of the plurality of output classifications, yielding a plurality of normalized expressions, each associated with one of the first plurality of molecular biomarkers, one of the plurality of output classifications, and one of the plurality of datasets. For each of the first plurality of molecular biomarkers, a pairwise comparison is performed between the normalized expressions associated with that molecular biomarker. Each pairwise comparison is between normalized expressions associated with a same output classification and a different dataset, thereby determining a transferability score for each of the plurality of molecular biomarkers. The first plurality of molecular biomarkers is ranked based on its transferability score. A second plurality of molecular biomarkers is generated from the first plurality of molecular biomarkers by applying a transferability score threshold to the first plurality of molecular biomarkers.

In some embodiments, each of the first plurality of molecular biomarkers is a gene. In some embodiments, each of the first plurality of molecular biomarkers is a protein. In some embodiments, each signature comprises a plurality of synaptic weights. In some embodiments, each signature comprises a mapping function. In some embodiments, each output classification comprises a phenotype. In some embodiments, the phenotype is a disease phenotype. In some embodiments, said normalization comprises quantile normalization. In some embodiments, said normalization is to a predetermined reference distribution. In some embodiments, performing the pairwise comparison comprises computing a Kolmogorov-Smirnov statistic.

In some embodiments, determining the transferability score comprises computing a mean of the pairwise comparisons. In some embodiments, the plurality of datasets comprises at least one dataset derived from each of a plurality of platform technologies. In some embodiments, the platform technologies comprise microarrays and RNA-sequencing. In some embodiments, the platform technologies comprise mass spectrometry, ELISA, antibody arrays, peptide fingerprinting, and/or protein barcoding. In some embodiments, each of the plurality of datasets are derived from the same biological samples.

According to embodiments of the present disclosure, methods of and computer program products for evaluating the robustness and transferability of predictive signatures across datasets are provided. In various embodiments, a method reads at least one signature. Each signature relates a first plurality of molecular biomarkers to one of a plurality of output classifications. For each of a plurality of datasets, each of the pair of datasets are derived from different platform technologies and from the biological samples, and a correlation coefficient for each of the first plurality of molecular biomarkers between the pair of datasets is determined. For each of the of the plurality of output classifications, a classification-specific correlation coefficient for each of the first plurality of molecular biomarkers between the pair of datasets is determined. The first plurality of molecular biomarkers is ranked based on each's correlation coefficient and classification-specific correlation coefficient. A second plurality of molecular biomarkers is generated from the first plurality of molecular biomarkers. A transferrable signature is provided relating the second plurality of molecular biomarkers to the first of the plurality of output classifications.

### BRIEF DESCRIPTION OF THE SEVERAL VIEWS OF THE DRAWINGS

FIGS. 1A-B illustrate exemplary groups of molecular biomarkers and associated groupings according to embodiments of the present disclosure.

FIGS. 2A-B illustrate RNA extraction and quantification of gene expression according to embodiments of the present disclosure.

FIG. 3 illustrates a method to ensure gene transferability according to embodiments of the present disclosure.

FIG. 4 illustrates the impact of quantile transformation on the distribution of expression values across samples in a given dataset according to embodiments of the present disclosure.

FIG. 5A-C illustrates the distribution of exemplary gene expression values grouped by phenotype labels according to embodiments of the present disclosure.

FIG. 6 illustrates the comparison between phenotype labels and datasets according to embodiments of the present disclosure.

FIG. 7 illustrates a pairwise Kolmogorov-Smirnov statistic according to embodiments of the present disclosure.

FIG. 8 the computation of a metric for feature transferability according to embodiments of the present disclosure.

FIG. 9 is a graph of cumulative probability, reflecting sorting genes by rank according to embodiments of the present disclosure.

FIG. 10 is a flowchart illustrating a method of determining feature transferability according to embodiments of the present disclosure.

FIG. 11 is a sample-wise rank plot of Spearman correlation coefficients between microarray and RNA-seq TPM expressions according to embodiments of the present disclosure.

FIG. 12 is a rank plot with Spearman correlation coefficient as transferability metric between microarray and RNA-seq TPM expressions according to embodiments of the present disclosure.

FIGS. 13A-B are rank plots of genes using Spearman correlation coefficient as the transferability metric between microarray and RNA-seq TPM expressions according to embodiments of the present disclosure.

FIG. 14 is a plot of the Spearman correlation coefficient between microarray and RNA-seq TPM expressions according to embodiments of the present disclosure.

FIGS. 15A-B are plots of an exemplary transferability statistic by gene rank according to embodiments of the present disclosure.

FIG. 16A-B are plots of an exemplary transferability statistic relative to gene rank according to embodiments of the present disclosure.

FIG. 17 is a plot of an exemplary transferability statistic relative to gene rank according to embodiments of the present disclosure.

FIG. 18 is a plot of an exemplary transferability statistic relative to gene rank according to embodiments of the present disclosure.

FIG. 19 is a plot of an exemplary transferability statistic relative to gene rank according to embodiments of the present disclosure.

FIG. 20 is a plot of an exemplary transferability statistic relative to gene rank according to embodiments of the present disclosure.

FIG. 21 depicts a computing node according to an embodiment of the present disclosure.

### DETAILED DESCRIPTION

A gene signature (or gene expression signature) is a single or combined group of genes in a cell with a uniquely characteristic pattern of gene expression that occurs as a result of an altered or unaltered biological process or pathogenic medical condition. A gene signature further requires the relationships between genes to be defined by some set of parameters, weights, values or rules.

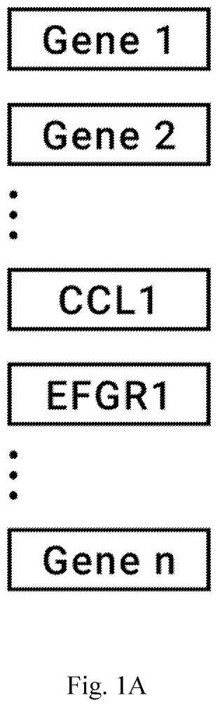

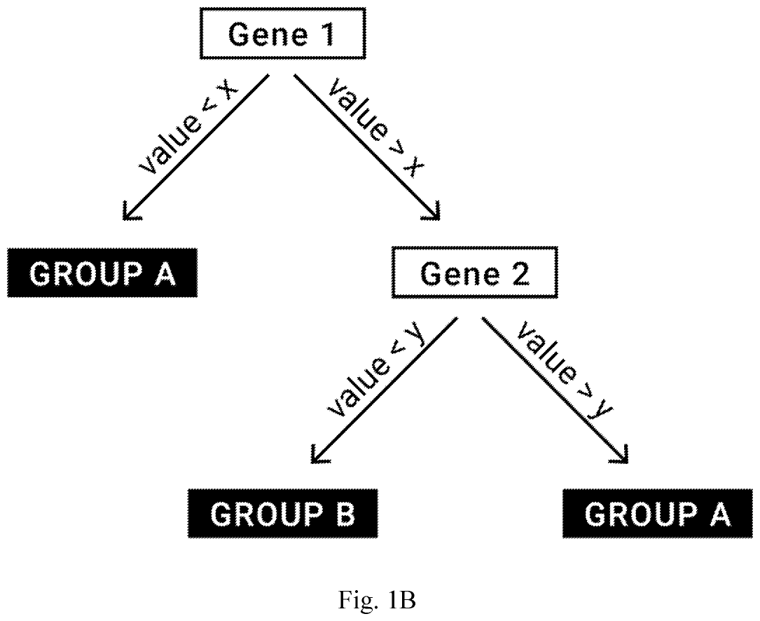

FIG. 1 illustrates these relationships. In FIG. 1A, an exemplary group of genes is illustrated. In FIG. 1B, a tree is provided that relates several exemplary genes to groups of interest via exemplary value.

Gene signatures are important to precision medicine, where gene signatures for a particular disease may be used as biomarkers, with utility to diagnose disease presence, classify disease type, and predict which patients are most likely to respond to a particular treatment, among other applications.

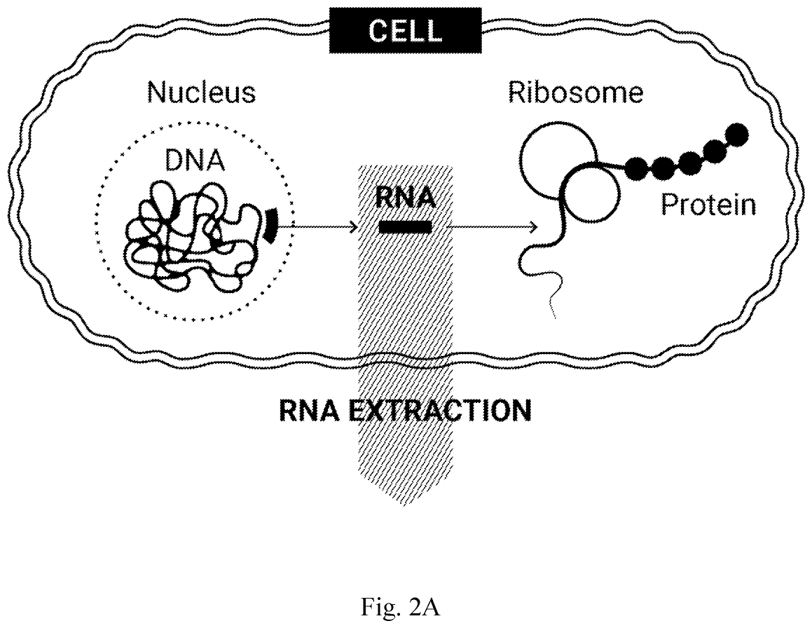

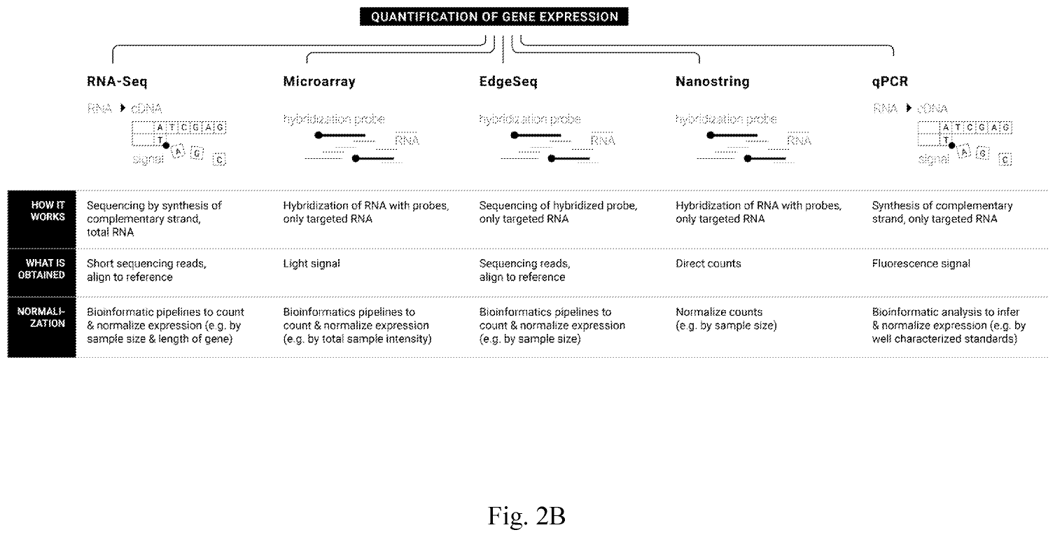

Gene signatures may be defined from datasets that measure gene expression—typically messenger RNA (mRNA) abundance—from biological samples. FIG. 2A illustrates the extraction of RNA from a cell. These may include experimental samples or patient derived samples, e.g., cells collected from a blood draw or tumor biopsy. Various mathematical approaches—within the fields of bioinformatics and biostatistics—may be used to define a gene signature on a particular dataset. Gene signatures may be generated using software tools like GSEA (Gene Set Enrichment Analysis), or via differential gene expression analysis or pathway analysis. Such tools depend on specific gene expression datasets as starting points. Alternatively, genes may be manually enumerated based on hypothesized mechanism of action.

Gene expression datasets may be generated from platform technologies such as microarrays or RNA-sequencing, or derivations thereof. FIG. 2B illustrates several approaches to quantifying gene expression once genetic materials have been extracted. However, a gene signature defined on one dataset will not necessarily display the same distribution or pattern of expressions when considered on other datasets. Several factors may, alone or in conjunction, limit the ability to transfer gene signatures between datasets, e.g.:
1. The processing of raw biological samples into sequencing libraries can introduce inconsistencies and biases, stemming from material handling, library chemistry, composition, etc.;
2. The sequencing or array platform technology used to generate the data can create incompatibilities in direct data comparison;
3. Demographics (such as age, gender), prior treatment, or experimental characteristics of the patient/biological samples can introduce confounders;
4. General batch effects stemming from unintended variation in any of the above or other factors.

Thus, a gene signature cannot be applied to a different dataset and be expected to retain its utility without taking steps to ensure its applicability to that new dataset. In other words, a gene signature is not transferable from one dataset to another without evaluating and correcting for transferability.

This creates a problem for the approval and commercialization of diagnostic, prognostic and predictive gene signatures. Without the ability to generalize a gene signature to newly generated datasets (e.g., new patient samples), a gene signature would be rendered practically useless and certainly unworthy of regulatory approval or clinical application.

Approaches to this problem may be separated into manual and semi-manual approaches. The former rely on curation by domain experts to perform sanity checks and smell tests (that is, experience driven heuristics) on the results when a gene signature is transferred to a new dataset. This is exceedingly subjective and prone to error and bias. Further, such manual approaches cannot be applied at a commercial scale, nor are they suitable for regulatory approval of a diagnostic product. Alternatively, various mathematical approaches may be employed to reduce this reliance on biased human inputs. For example, a Principal Component Analysis (PCA)-based approach may be used to reduce a gene signature to a summary score that can be compared across datasets. Such methods, however, have a fundamental limitation that complex signatures, signatures describing multiple events, do not work well with PCA. In the context of complex diseases like cancer, often times a gene signature results from the interplay of many cellular, genetic and chemical entities, and thus PCA-based methods are likely not appropriate. Another approach uses a zero-sum regression signature learned on high-content data, in which the weights are retained from one dataset to the next.

Thus, precision medicine requires a method for transferring gene signatures from one dataset to another that is robust to the data-generation technology and patient sample source. Such methods should minimize the assumptions of data provenance and distribution characteristics, and should be applicable to gene signatures that represent complex biology.

To address these and other shortcomings of alternative approaches, the present disclosure provides supervised learning systems and methods that autonomously constructs a gene signature by training a classification or regression model on one or more gene expression datasets—such that the model is agnostic of the dataset technology, processing of raw biological samples, and other batch effects—and can be applied to other distinct datasets for the prediction task.

In various embodiments, it is assumed that gene expression has been measured using any transcriptomics platform technology, including but not limited to RNA-sequencing by Illumina or IonTorrent, HTG Edge-seq, Nanostring, qPCR, or microarray. It is further assumed that expression values for each gene in a particular gene set (or all genes in the genome) have been computed using standard bioinformatics programs (e.g., RNA-Seq methods and pipelines known in the art, including those provided by Genialis, Inc.).

Likewise, while various examples provided below pertain to gene expression data, the techniques described herein are generally applicable to molecular biomarkers including genes, proteins, and metabolites. For example, in embodiments directed to proteomic data, it is assumed that protein expression has been measured using any proteomics platform technology, including but not limited to mass spectrometry, ELISA, antibody arrays, peptide fingerprinting, protein barcoding or other similar methods for inferring protein sequences of a plurality of proteins from a biological sample. It is further assumed that values for each protein in a particular signature (or all proteins in the proteome) have been computed using standard bioinformatics programs (e.g., proteomics methods and pipelines known in the art, including those provided by Genialis, Inc.).

In various embodiments of supervised learning systems and methods, the inputs include expression matrices from the datasets, and a list of genes (e.g., up to several hundred genes) or other molecular biomarkers such as proteins. The output is a gene signature function or other signature function related to a molecular biomarker.

The signature function is inferred from labeled training data consisting of a set of training samples. Each sample is a pair consisting of an input object (e.g., a vector of gene expressions) and a desired output value (which can be discrete or continuous). It will be appreciated that one or more continuous value output may be converted to a classification by binning, thresholding, winner-take-all, and various other methods. The training data is analyzed to produce an inferred function, which can be used for mapping new samples from other distinct datasets. The inferred gene signature function may take a variety of forms according to the particular machine learning method employed. For example, the signature function may be a matrix operator that is applicable to an input expression matrix from a sample. In another example, the signature function may be a set of synaptic weights for an artificial neural network.

In various embodiments, supervised learning techniques are employed such as artificial neural networks, random forests, support vector machines, and logistic regression. It will be appreciated that a variety of additional supervised learning techniques are suitable for use according to the present disclosure. Ensemble techniques such as stacking are used in various embodiments to improve accuracy. Special care must be taken to avoid overfitting, especially in parameter tuning. Training and test datasets should include distinct, non-overlapping sets of samples. Samples may be partitioned using cross-validation, bagging (bootstrap Aggregation) or other approaches.

In some embodiments, a feature vector is provided to a learning system. Based on the input features, the learning system generates one or more outputs. In some embodiments, the output of the learning system is a feature vector.

In some embodiments, the learning system comprises a SVM. In other embodiments, the learning system comprises an artificial neural network. In some embodiments, the learning system is pre-trained using training data. In some embodiments training data is retrospective data. In some embodiments, the retrospective data is stored in a data store. In some embodiments, the learning system may be additionally trained through manual curation of previously generated outputs.

In some embodiments, the learning system, is a trained classifier. In some embodiments, the trained classifier is a random decision forest. However, it will be appreciated that a variety of other classifiers are suitable for use according to the present disclosure, including linear classifiers, support vector machines (SVM), or neural networks such as recurrent neural networks (RNN).

Suitable artificial neural networks include but are not limited to a feedforward neural network, a radial basis function network, a self-organizing map, learning vector quantization, a recurrent neural network, a Hopfield network, a Boltzmann machine, an echo state network, long short term memory, a bi-directional recurrent neural network, a hierarchical recurrent neural network, a stochastic neural network, a modular neural network, an associative neural network, a deep neural network, a deep belief network, a convolutional neural networks, a convolutional deep belief network, a large memory storage and retrieval neural network, a deep Boltzmann machine, a deep stacking network, a tensor deep stacking network, a spike and slab restricted Boltzmann machine, a compound hierarchical-deep model, a deep coding network, a multilayer kernel machine, or a deep Q-network.

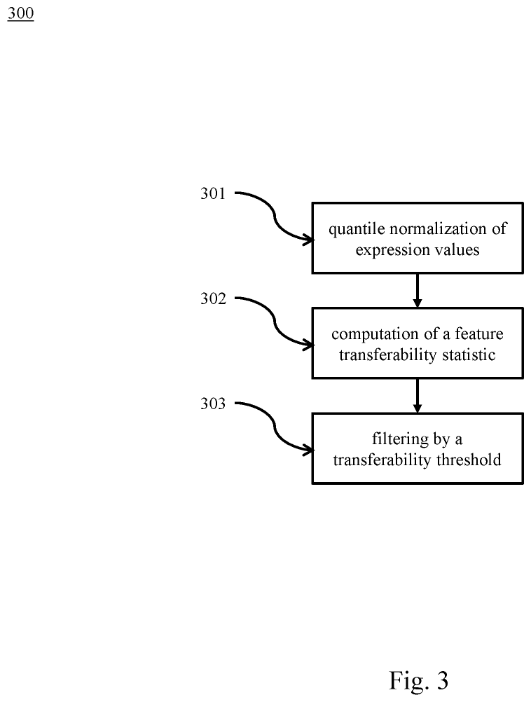

Referring to FIG. 3, a method to ensure gene transferability is illustrated according to embodiments of the present disclosure. At 301, quantile normalization of expression values is performed. At 302, computation of a feature transferability statistic is performed. At 303, features (e.g., genes) are filtered by a transferability threshold.

For the purposes of illustration, the following example draws on exemplary data. It will be appreciated that the present disclosure is applicable to a variety of datasets and labels, and that this example is illustrative rather than limiting. In this example, gene expression data are taken from the following datasets: Asian Cancer Research Group (ACRG); The Cancer Genome Atlas (TCGA); and Singapore Cohort (SING).

The individual samples in these datasets are further labeled as the following phenotype classes: Phenotype 1, Phenotype 2, Phenotype 3, Phenotype 4.

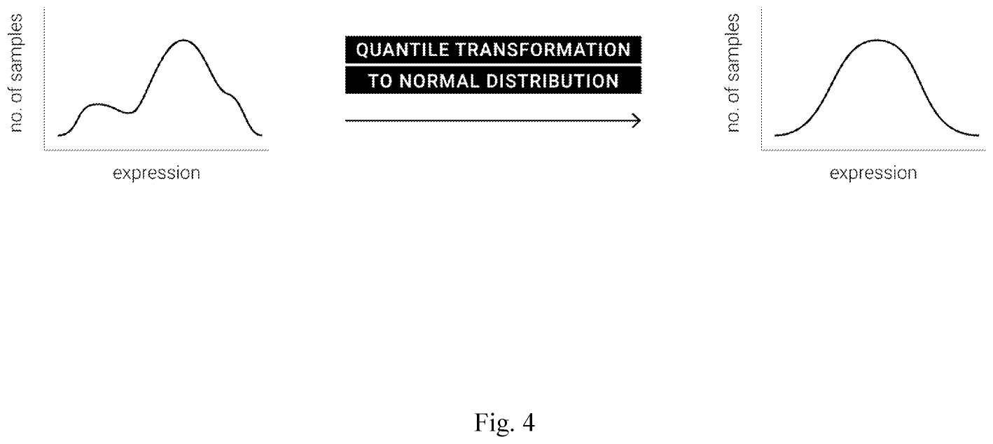

Quantile normalization is a technique for making two distributions identical in statistical properties. FIG. 4 illustrates the impact of quantile transformation on the distribution of expression values across samples in a given dataset. Datasets are normalized against a reference distribution which is one of the standard statistical distributions such as the Uniform distribution, the Gaussian distribution, or the Poisson distribution. The reference distribution can be generated randomly or from taking regular samples from the cumulative distribution function of the distribution. Any reference distribution can be used.

All gene expression datasets are in turn normalized to the same reference distribution. The transformation is applied on each feature (expression values of one gene) independently. First an estimate of the cumulative distribution function of a feature is used to map the original values to a uniform distribution. The obtained values are then mapped to the desired output distribution using the associated quantile function.

The robustness of the procedure increases logarithmically with the number of samples. Several tens of samples (about 30 or more) per dataset are required to guarantee base-level performance of the gene signature. The overall performance of gene signature gradually increases and flattens as the number of samples being quantile normalized reach mid hundreds.

In various embodiments, quantile normalization is used as a preprocessing procedure in supervised learning, thus special care must be taken to avoid overfitting. The quantile normalization parameters should be fitted on the training set of samples, and then used to transform the testing and validation samples. The testing and validation samples must be excluded from fitting the parameters of quantile normalization.

Transferable features (genes) should have a similar distribution of gene expression values between datasets given the target variable (phenotype or outcome label). Some, however, are vastly different and should be excluded from the gene signature. The difference may be attributed to technology (e.g., RNA-seq vs. microarray), experiment bias, population bias, and other effects.

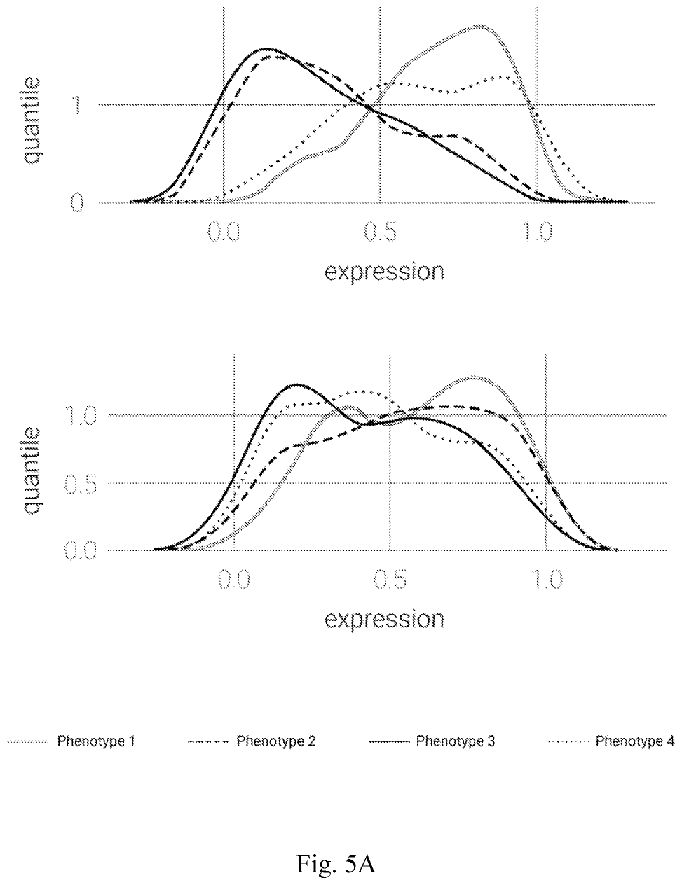

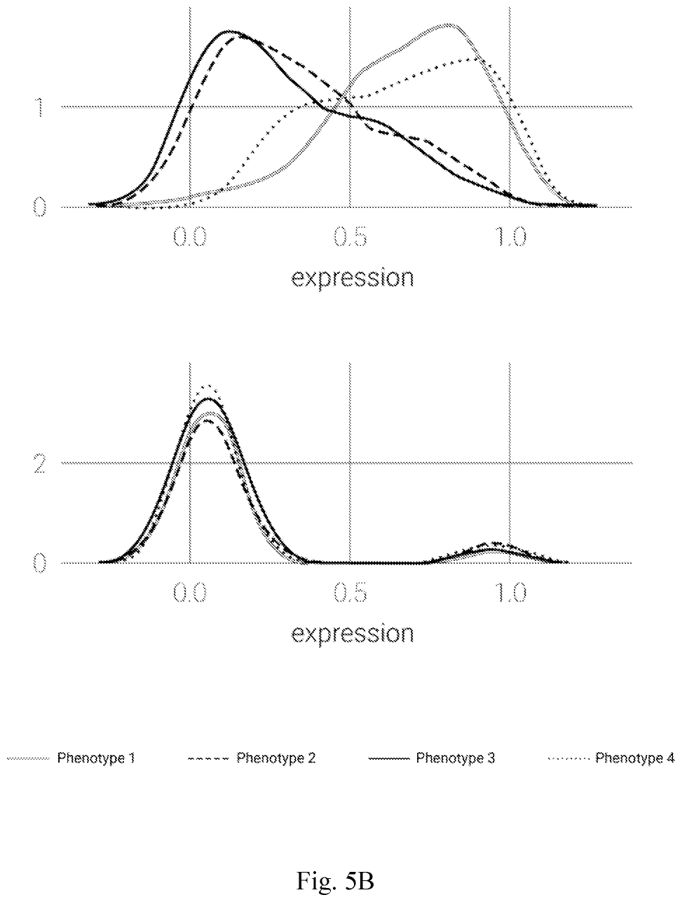

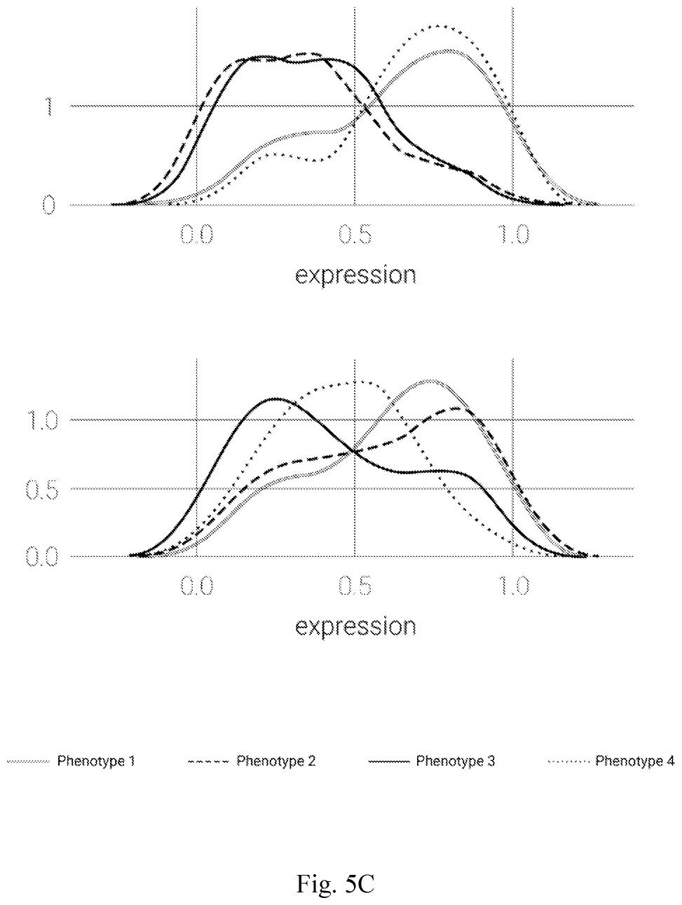

In FIGS. 5A-C, the distribution of exemplary gene expression values are grouped by the four phenotype labels (in legend). First row: gene CCL3, second row: gene IFNA2. FIGS. 5A-C represent ACRG, TCGA and SING datasets, respectively. The expression values are quantile normalized to uniform distribution (within each dataset separately). The distribution of gene expression estimates of CCL3 are consistent between datasets, but are inconsistent for IFNA2.

The present disclosure provides a metric for feature transferability defined as a reduced set of test statistics obtained from pairwise comparisons of distributions of gene expression datasets.

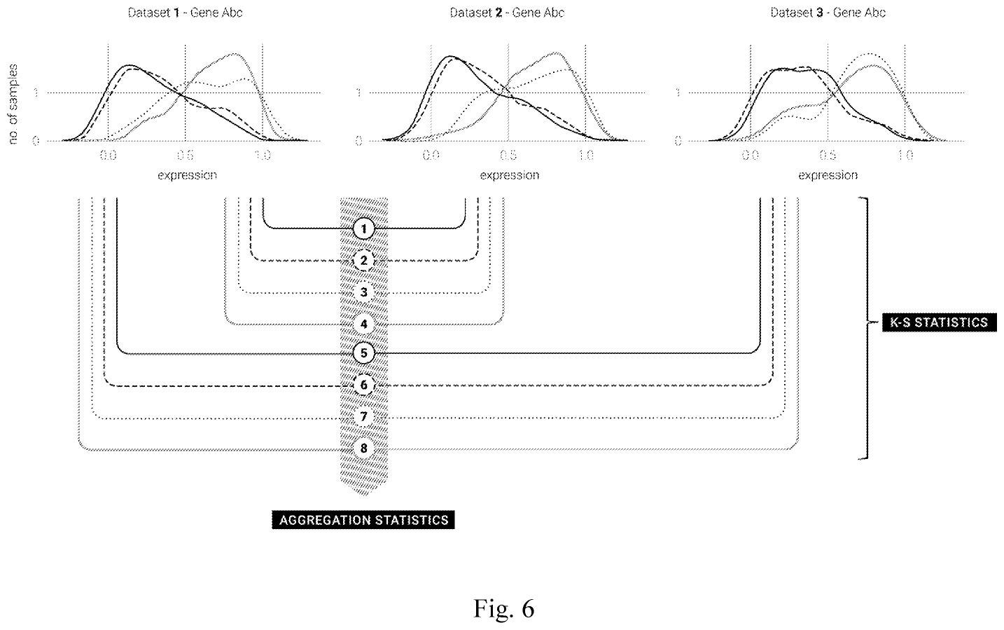

The test statistics should be selected based on whether the target variable is categorical, continuous, or other. In the illustrative case below, metadata are categorical (phenotypes 1 to 4). Feature transferability is derived from an aggregation—e.g., the arithmetic mean—of pairwise Kolmogorov-Smirnov tests of phenotype-specific distributions of gene expressions between datasets. This process is illustrated in FIG. 6, where the four phenotype labels are compared in a pairwise fashion between the first and second dataset and between the first and third dataset. Aggregation may also be achieved by considering the median or min-max range characteristics, and the most appropriate type of aggregation may be calculated empirically.

The Kolmogorov-Smirnov (K-S) test is a nonparametric test of the equality of continuous, one-dimensional probability distributions that quantifies a distance between the empirical distribution functions of two samples. The K-S statistic is defined as a maximum difference between two joint cumulative distribution functions. The arithmetic mean of K-S statistic denotes the average distance between the distributions of expression values grouped by the four phenotypes.

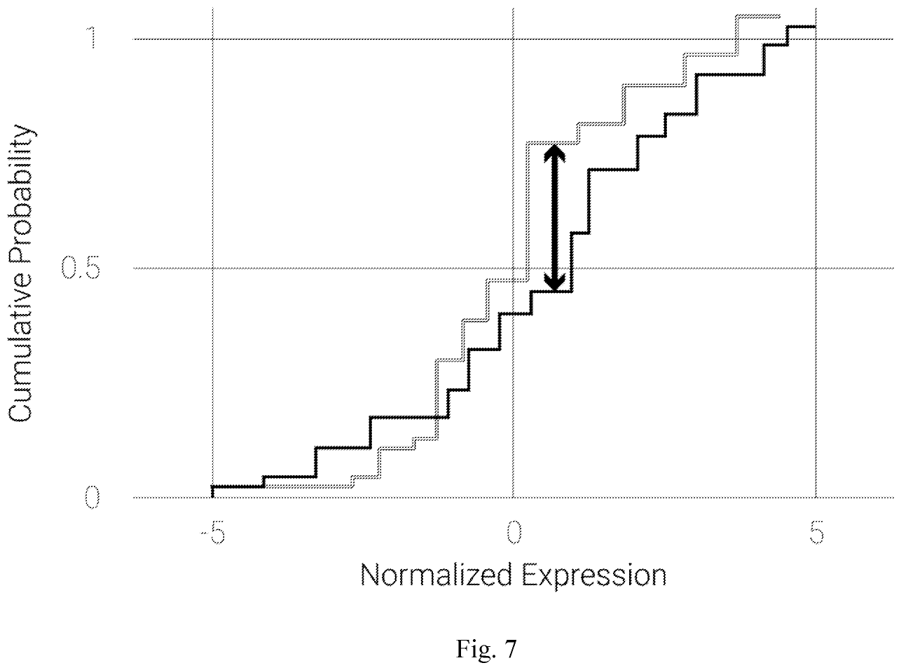

FIG. 7 illustrates the pairwise Kolmogorov-Smirnov statistic. Light and dark lines each correspond to an empirical distribution function, and the black arrow marks the difference in distribution captured by the K-S statistic.

Using this metric, one can reduce the dataset bias by removing the features with inconsistent distribution of gene expressions. For each gene, multiple K-S statistics are computed, one for each combination of phenotype and dataset pair. In order to obtain a single transferability score for each gene, K-S statistics need to be aggregated across phenotypes and dataset pairs. Among common aggregation methods, arithmetic mean worked well for these illustrative datasets. However, it will be appreciated that alternative methods such as median, min and max may be used in some embodiments.

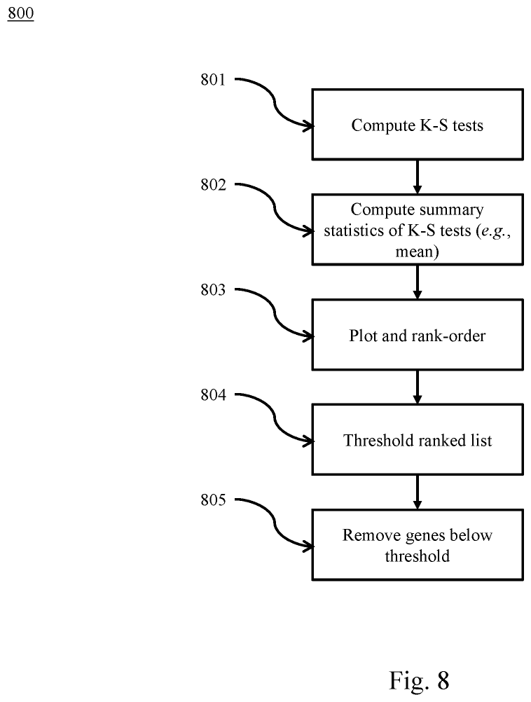

Referring to FIG. 8, the computation of a metric for feature transferability is illustrated according to embodiments of the present disclosure. At 801, a battery of K-S tests is computed for: a) Gene expression values across all phenotype/outcome classes; and b) two dataset pairs (ACRG-TCGA, TCGA-SING). In this example case, the phenotype/output classes include: Phenotype 1, Phenotype 2, Phenotype 3, Phenotype 4. At 802, the average of the eight K-S tests is computed.

At 803, the K-S statistic is plotted & rank-ordered for all genes in a particular signature. At 804, the ranked gene list is thresholded. In some embodiments, thresholding is performed by selecting the point just prior to the start of the rapidly increasing tail of the K-S statistic (a point on the X-axis). Genes with low K-S statistics (ranked closest to 1) are considered most transferable. In some embodiments, thresholding is performed by converting the K-S statistics into p-values using standard conversion tables and selecting a p-value cut-off (setting the threshold on the y-axis and not the x-axis). After correcting for multiple hypothesis testing, one may confidently select a useful p-value threshold.

At 805, the genes that do not meet the K-S or p-value threshold are removed from the signature.

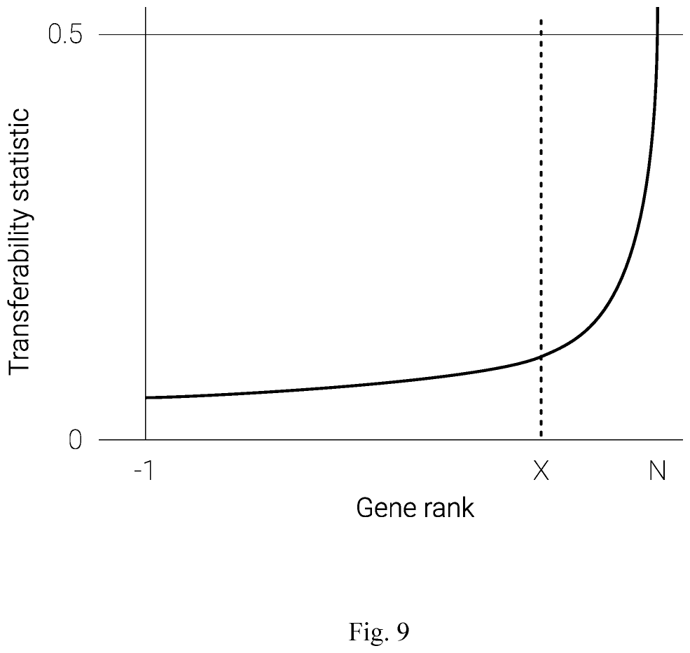

Referring to FIG. 9, a graph of cumulative probability is provided, reflecting sorting genes by rank. In this case, the threshold static values is set at gene rank 98 (out of 125 genes in the example signature) based on the rapid increase in curve slope at values greater than 98. Thus, one would categorize genes ranked 99 to 125 as “non-transferable” and remove them from the model.

Threshold values may be inferred automatically by determining the second derivative of the transferability curve to identify an inflection point. It will be appreciated that a variety of techniques are known for locating such a threshold. For example, in some embodiments, an average is taken using a sliding window. In some embodiments, the threshold is set according to a predetermined change in slope of the curve. In some embodiments, the threshold is determined empirically based on the distribution of changes in slope.

The methods described herein may be applied in any pharmaceutical or diagnostic R&D setting in which gene expression data are being evaluated for predictive potential. For example, transferable gene signatures output from this method could form the basis of a companion diagnostic (Cdx) or Lab Developed Test (LDT) for a drug. Thus, a transferable gene signature could form the basis for an approved diagnostic test deployed at the point of care by clinical practitioners. Alternatively, a transferable gene signature might constitute a list of potential drug targets for early drug discovery R&D. Because the transferable gene signature is robust to patient demographics, it may be used to assess drug repositioning. Lastly, one may use the method to guide indication expansion, that is, identifying new disease areas for which to test the efficacy of a particular drug or therapy.

As set out above, methods are provided for determining whether the genes of a gene expression signature that serve as features of a model behave consistently across datasets having different derivation (e.g., different data generating technology platforms, diseases, patient cohorts, etc.).

In some cases, gene expression data generated by two different technology platforms will be available for the same biospecimen. For example, certain cell line libraries (e.g., the Cancer Cell Line Encyclopedia (CCLE) by the Broad/Novartis) have been profiled by both gene expression microarrays and by RNA-sequencing. Likewise, archival tumor biopsies that were previously analyzed by microarray may be analyzed anew by RNA sequencing (e.g., The Cancer Genome Atlas (TCGA), among others). A challenge to applying a gene signature or predictive model derived from the microarray data to newly generated RNAseq data is determining whether the gene features are transferable across these technologies. Overcoming this challenge is essential to making use of potentially valuable historical datasets, or any data and analyses performed on previous generation expression technologies. Given the rapid pace of change in ‘omics profiling, important datasets are at risk of becoming obsolete every few years. They can be revived and carried forward using the methods described herein for determining feature transferability.

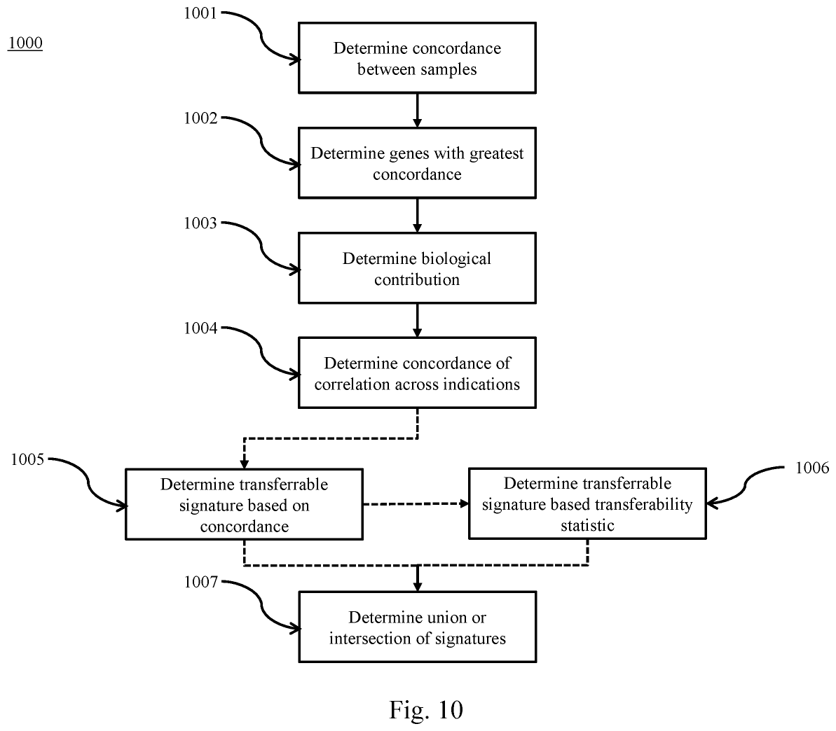

Referring to FIG. 10, an exemplary method is provided to, given a gene signature and a dataset of paired gene expressions generated by microarray and RNA-seq, assess the impact of technology platform and biological variation (e.g., disease type) on feature transferability.

At 1001, the concordance between samples analyzed by different technology platforms is determined. For each pair of samples, Spearman correlation coefficients are computed between microarray and RNA-seq expressions of signature genes. The samples are sorted by Spearman correlation coefficient in descending order. For each pair of samples the Spearman correlation coefficient is plotted as a function of sample rank. Samples with concordance below a certain threshold may be excluded, or examined individually to determine the source of variation. At this step, all samples are treated together regardless of disease type.

An exemplary dataset, includes a signature of 170 genes, and microarray and RNA-seq data from 140 pairs of cell line samples from the CCLE. These 140 sample pairs correspond to three different cancer types: 110 gastric cancer, 22 sarcoma, and 8 mesothelioma.

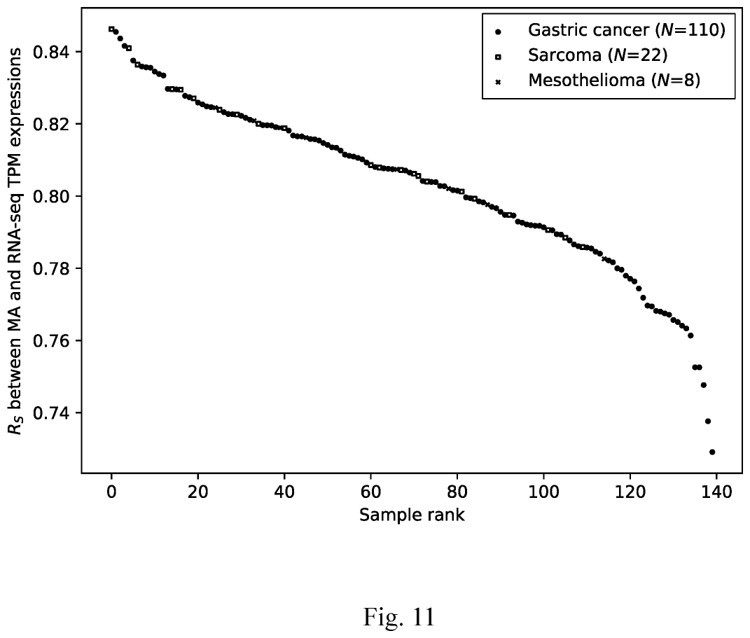

Referring to FIG. 11, a sample-wise rank plot is provided based on Spearman correlation coefficient between microarray and RNA-seq TPM (Transcript Per Million normalization) expressions. This includes all considered disease types: gastric cancer, sarcoma, and mesothelioma. This analysis reveals relatively high concordance between microarray and RNA-seq TPM expressions for nearly all samples, and from all included disease types. Spearman correlation coefficients of samples are mostly near R_{S}=0.8, in line with industry standard.

Upon visual inspection, one could consider removing samples below 0.75 since these drop off markedly from the rest. However, it will be appreciated that a variety of statistical methods may be used to determine the cutoff value, as set forth above.

At 1002, the genes that show greatest concordance across all sample pairs are determined. For each gene, Spearman correlation coefficients are computed between microarray and RNA-seq expressions of paired samples. Genes are sorted by Spearman correlation coefficient in descending order. For each gene, the Spearman correlation coefficient is plotted as a function of gene rank.

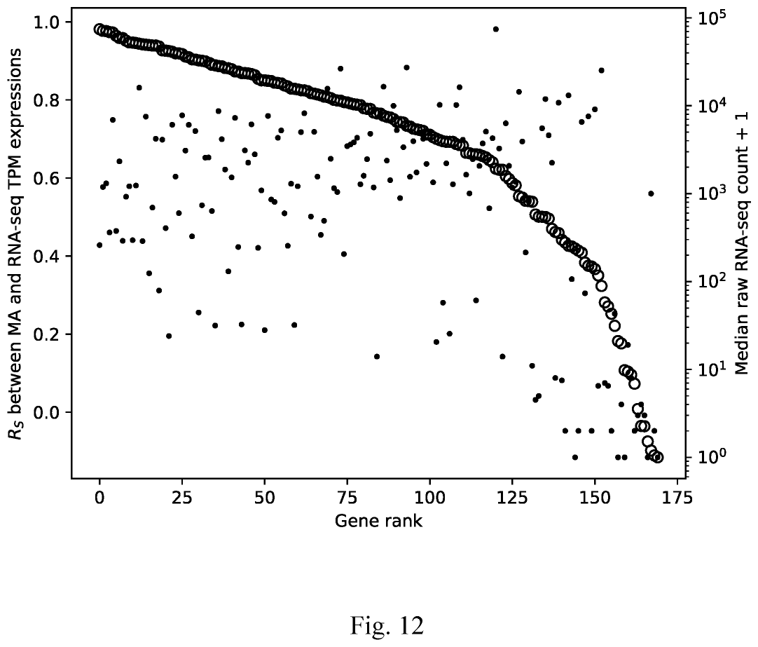

Referring to FIG. 12, a rank plot is provided of 170 genes with Spearman correlation coefficient as transferability metric between microarray and RNA-seq TPM expressions. Each point represents a gene. Each correlation coefficient is computed across all sample pairs (in this example, gastric cancer, sarcoma and mesothelioma subjects (140 in total)). The lefty-axis (large circles) corresponds to the Spearman correlation coefficient between microarray and RNA-seq TPM expressions computed across the aforementioned samples. The righty-axis (small circles) corresponds to the median raw RNA-seq count +1 computed across the aforementioned samples.

Gene-wise correlation between expression derived from microarray and RNA-seq decreases linearly for about top 125 genes after which it rapidly drops off. Genes with the lowest rank have the largest correlation (in this dataset, CXCL8 (R_{S}=0.98)). A threshold may be set on the left vertical axis where the linear slope changes (to supra-linear or exponential decay). In the above example, this inflection point occurs around R_{S}=0.60, thus all genes with rank >˜125 could be removed from the analysis.

Correlation between microarray and RNA-seq TPM expressions can be partially explained by the level of expression of genes. Poorly expressed genes with median raw RNA-seq count below 10 mostly show correlation R_{S}<0.2. On the other hand, expressions of genes with median raw count over 100 often correlate well (R_{S}>0.6) between microarrays and RNA-seq. Thus, this overlay can enable the determination of a minimum gene expression threshold below which certain genes may be excluded.

At 1003, the contribution of biological factors (as opposed to technology platform) to gene/sample rank is determined. For each gene, the Spearman correlation coefficient is computed between microarray and RNA-seq expressions of paired samples, separately for each disease. In this example, the diseases covered are: gastric cancer, sarcoma and mesothelioma. Genes are sorted by Spearman correlation coefficient in descending order. For each gene, the Spearman correlation coefficient is plotted as a function of gene rank of the disease type with the most samples (in this case, gastric cancer is the most prevalent type).

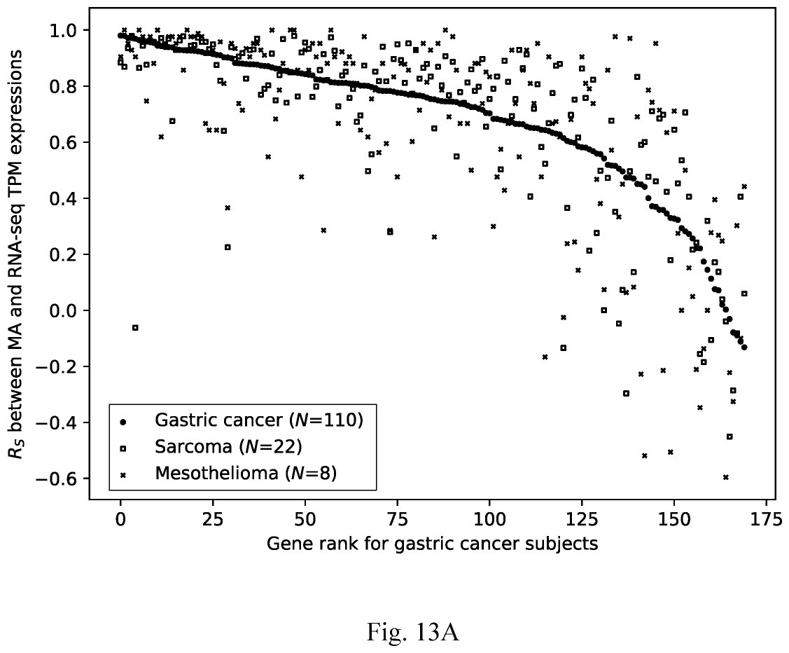

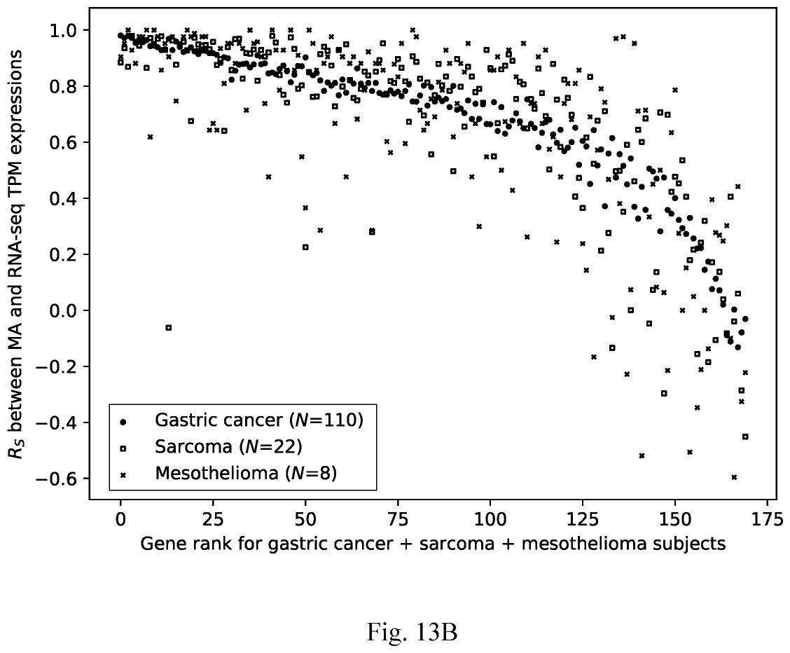

Referring to FIG. 13A, a rank plot is provided of genes using Spearman correlation coefficient as the transferability metric between microarray and RNA-seq TPM expressions. Each point represents a gene. Each correlation coefficient is computed across pairs separately for samples from each biological condition or disease (in this case, gastric cancer, sarcoma and mesothelioma).

The above computation of Spearman correlation coefficient is repeated, using gene rank based on all disease types rather than the most prevalent.

Referring to FIG. 13B, an alternative plot is provided, in which genes are ranked on the x-axis based on the correlation across subjects of all three indications instead of just the most prevalent.

The scatter in FIG. 13B relative to FIG. 13A indicates the extent to which variation in concordance is driven by biological condition. This is an important observation if the goal of gene signature development is to create a versatile feature set that can serve as a gene panel across conditions—e.g., a pan-cancer diagnostic.

At 1004, the concordance between correlation coefficients is examined across disease indications. For each gene, the Spearman correlation coefficient is computed between microarray and RNA-seq expressions of paired samples as in Step 1003. The correlation coefficient of samples representing conditions (B, C, . . . Z) are plotted as a function of correlation coefficient of condition A. In this example, B=Sarcoma, C=Mesothelioma, and A=Gastric cancer. If one of these conditions is clearly most prevalent, it can serve as the independent variable. If the conditions are more evenly distributed, the analysis should be repeated, rotating which condition serves as the independent variable.

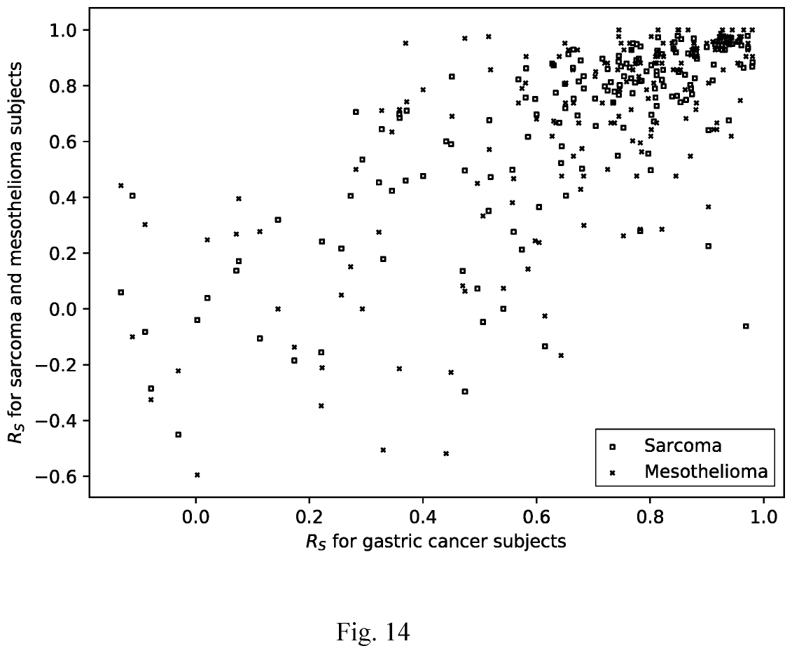

Referring to FIG. 14, the Spearman correlation coefficient between microarray and RNA-seq TPM expressions of conditions B & C (sarcoma and mesothelioma) is shown as a function of the same correlation coefficient for condition A (gastric cancer). Each point corresponds to a gene.

Genes that are most consistently highly correlated between sample pairs cluster in the upper right. A box drawn at (X,Y=0.6,0.6) will gate the features that are informative across biological conditions (e.g., diseases). This analysis confirms the thresholding approach in step 1002.

In some embodiments, the most consistently highly correlated genes (or other molecular biomarkers) in an input signature are retained in order to derive a transferrable signature at 1005. However, the concordance method described above may be combined with the transferability statistic (KS) method described above. For example, the transferability statistic may be computed at 1006 for each of the highly correlated biomarkers determines at 1005. Alternatively, signatures using each method may be computed in parallel at 1005, 1006 and then combined into an aggregate signature at 1007. The aggregate signature may be determined by taking the union or intersection of the two input signatures.

The expressions of each gene across all samples is quantile-transformed to a uniform distribution. For each gene, the Kolmogorov-Smirnov test statistic is computed in all sample pairs for all biological conditions (e.g., gastric cancer, sarcoma and mesothelioma) using distributions of quantile-normalized expressions. The genes are sorted by Kolmogorov-Smirnov statistic in ascending order. For each gene and combination of disease indications, the Kolmogorov-Smirnov statistic is plotted as a function of gene rank.

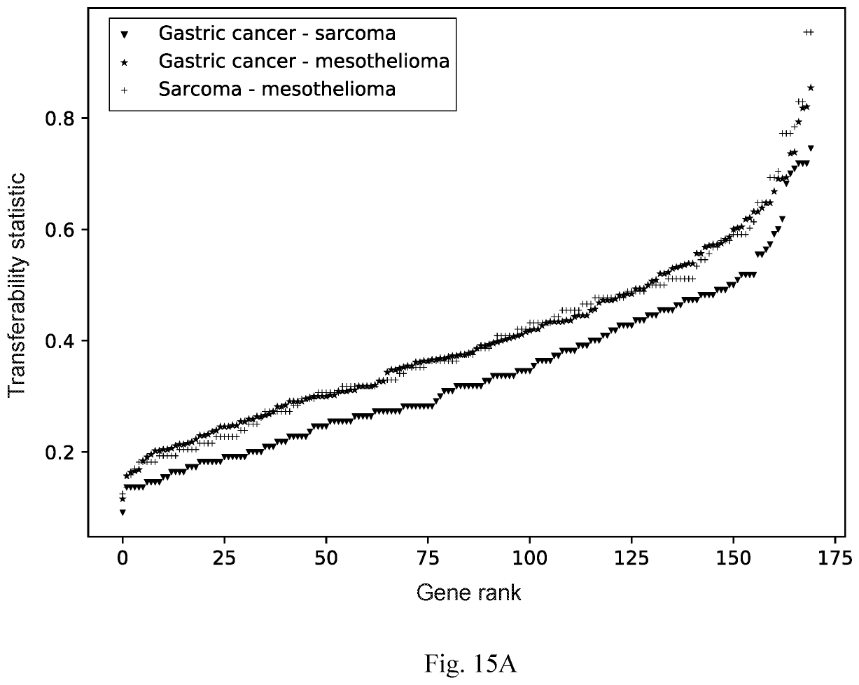

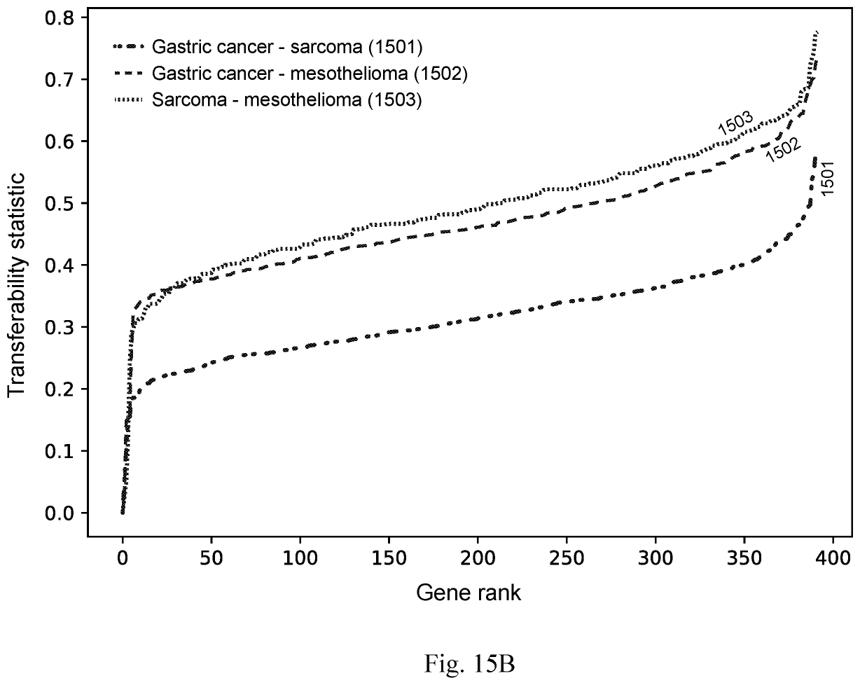

Referring to FIG. 15A, a plot is provided of the Kolmogorov-Smirnov statistic by gene rank. This shows the transferability of distribution of expressions by gene between A-B, A-C, and B-C (gastric cancer, sarcoma and mesothelioma) subsets of samples.

The best transferability of genes is consistently achieved between A-B (gastric cancer and sarcoma). Transferability between A-C (gastric cancer and mesothelioma) is similar to transferability between B-C (sarcoma and mesothelioma). The trend of K-S statistic as a function of gene rank is mostly linear. The value of K-S statistic grows quite rapidly into the regime where transferability is questionable at best (in this example, KS>0.5). As set forth above, instead of setting the cut-off based on the inflection point, it may be set based on a pre-determined or empirical transferability statistic value. In addition, it will be appreciated that the K-S statistic may be converted to a P-value or other probability in order to set the threshold.

Referring to FIG. 15B, a plot is provided of the Kolmogorov-Smirnov statistic by gene rank. This illustrates transferability of distribution of expressions by gene between A-B, A-C, and B-C (gastric cancer, sarcoma and mesothelioma) subsets of samples on an extended input gene set. The cross-disease transferability between gastric cancer and sarcoma is observed/confirmed on this expanded feature set.

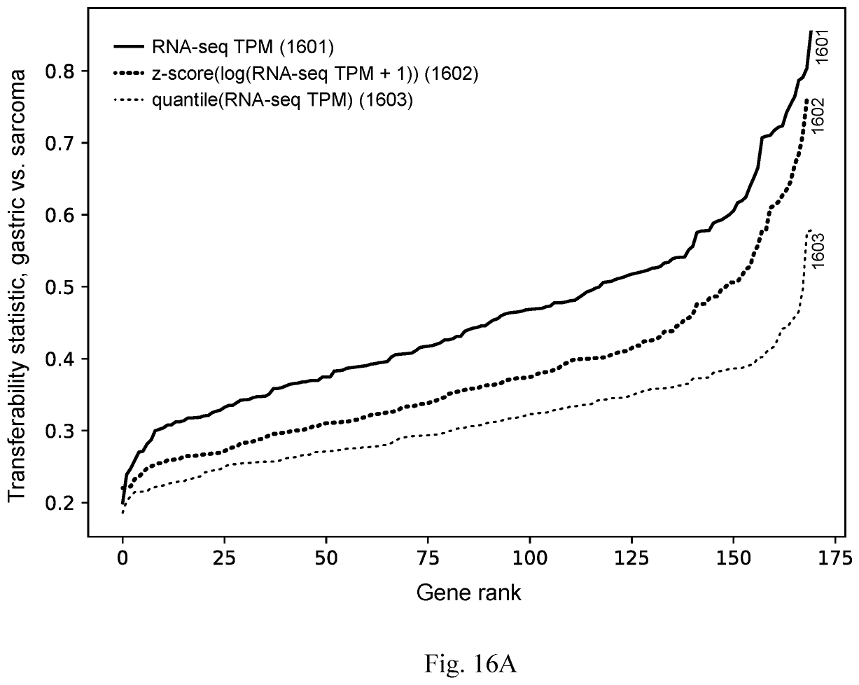

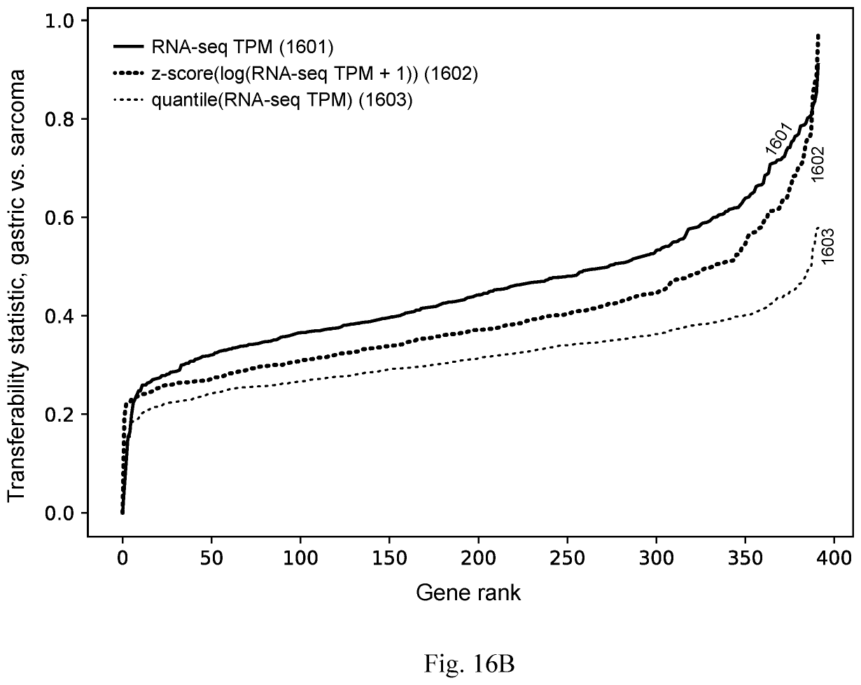

Referring to FIGS. 16A-B, evidence of the utility of quantile normalization is provided. In these examples, the same KS rank method is applied as described above, for A-B (gastric cancer-sarcoma) disease comparison. Three expression preprocessing methods are compared: TPM normalization, z-score(TPM+1) and quantile transformation of TPM-normalized expressions.

FIG. 16A shows the transferability of distribution of expressions by gene between gastric cancer and sarcoma for three expression preprocessing methods.

FIG. 16B shows the transferability of distribution of expressions by gene between gastric cancer and sarcoma for three expression preprocessing method, using an expanded feature set.

Quantile transformation (1603) displays superior performance followed by z-score (1602) and no preprocessing (1601). The above result can be recapitulated across all pairwise condition comparisons.

An additional utility of the method is to estimate transferability between samples of different diseases based on therapeutic phenotype. For example, one can ask whether genes that predict drug sensitivity are more transferable than genes that predict drug resistance. Thus, input samples are stratified by phenotype label, and the transferability statistic computed as before between two conditions (below, between gastric cancer and sarcoma).

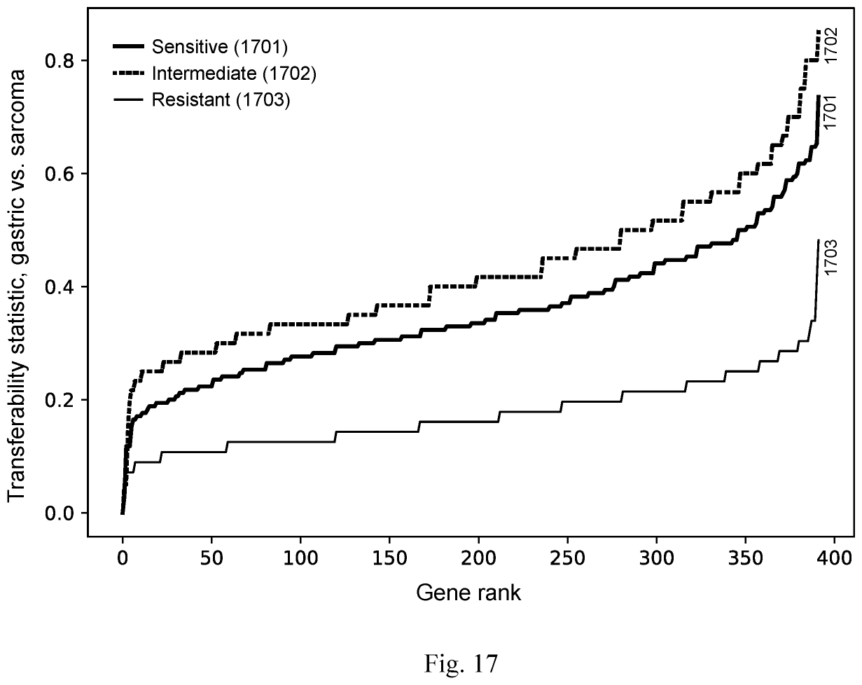

Referring to FIG. 17, a graph is provided illustrating the transferability of distribution of expressions by gene between gastric cancer and sarcoma for each response group of samples separately.

The observation that genes (features) are more transferable for cell lines of the “Resistant” phenotype suggests that the biological pathways responsible for drug resistance are conserved between disease conditions (gastric v sarcoma), whereas the biological pathways contributing to drug sensitivity are more heterogeneous.

In this way, the feature transferability method allows the inference of which drug response phenotype may be most confidently predicted from a given feature set.

As set out above, the feature transferability methods provided herein are broadly applicable. Several additional examples follow.

Transferability Across Data Generation Platforms

In a first example, transferability between Microarray and RNA-seq platforms derived from distinct patient subpopulations at different times and with different treatment histories are assessed.

The datasets used in this example were:
1) ACRG (Asian Cancer Research Group)
   Gastric cancer subjects (N=300) were second line or beyond, receiving prior chemotherapy and/or radiation
   Affymetrix microarray; GEO GSE62254, GSE62717; Cristescu et al 2015
2) TCGA (The Cancer Genome Atlas)
   Gastric cancer subjects (N=388) were a mixture of multiple lines of treatment
   RNA-seq; Data at portal.gdc.cancer.gov; Cancer Genome Atlas Research Network 2014
3) Singapore Cohort
   Gastric cancer subjects (N=192) were a mixture of multiple lines of treatment
   Affymetrix microarray platforms; GEI (GSE15459); Lei et al 2013

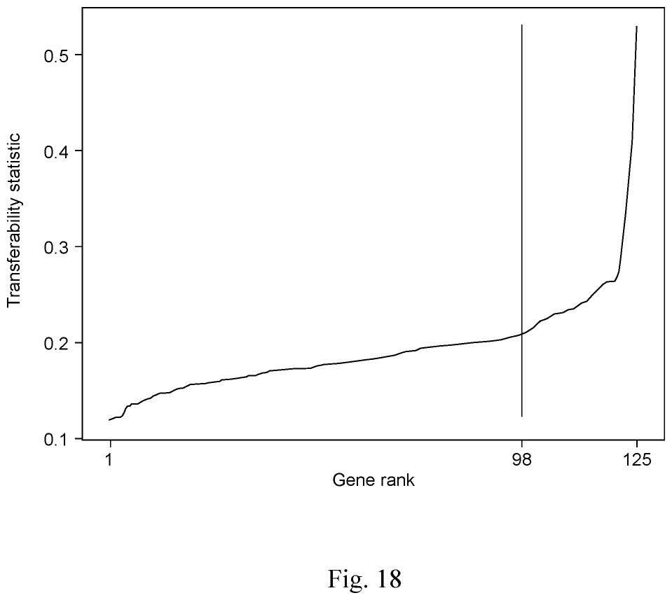

Referring to FIG. 18, a plot is provided of a K-S statistic versus gene rank. We computed the K-S statistics for 125 signature genes. When sorted by rank, one can observe an initial increase in the K-S statistic slope at rank 98. Thus, the remaining 27 genes may be deemed non-transferable and removed from the model.

Transferability Across Data Platform, Disease Tissue Type

In this example, transferability between Ovarian/gynecological and anti-VEGF datasets is assessed on the following axes—Platform: Microarray, exome RNA-seq, and total RNA-seq; Tissue types: ovarian/gynecological and gastric cancer.

The datasets used in this example were:
1) Proprietary clinical trial (anti-VEGF/DLL4 therapy, ovarian and gynecological cancers)
   Single arm phase 1b study of 4+ line platinum resistant patients with ovarian cancer treated with the combination of anti-VEGF/antiDLL4 bispecific plus paclitaxel
   RNA-seq (subset N=30); Data are unpublished
2) ACRG (Asian Cancer Research Group)
   Gastric cancer subjects (N=300) were second line or beyond, receiving prior chemotherapy and/or radiation
   Affymetrix microarray; GEO GSE62254, GSE62717; Cristescu et al 2015
3) Proprietary Gastric VEGF
   Subjects with gastric and GEJ cancer, mixed prior treatment history, 100% Asian demographic
   Treated with anti-VEGF ramucirumab
   RNA-seq (N=48); Data are unpublished
4) ICON7
   Subjects with Ovarian cancer
   Treated with chemotherapy+bevacizumab (anti-VEGF)
   Microarray (N=380); GEO accession GSE140082)

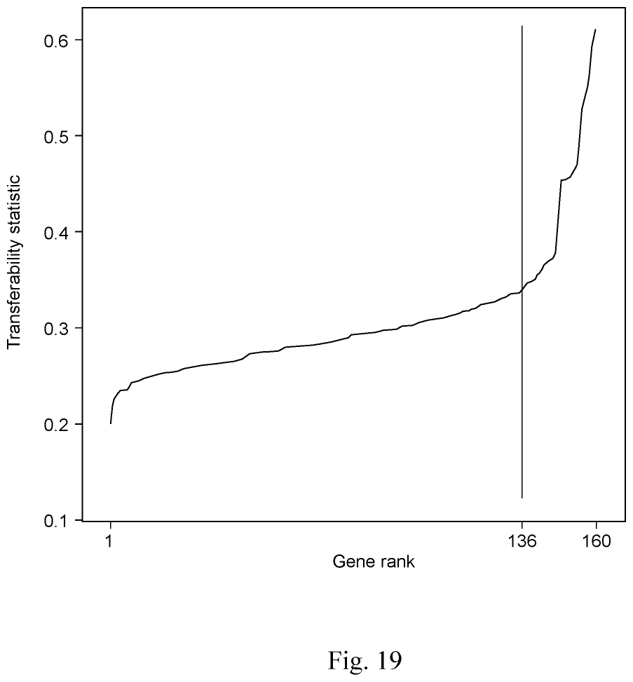

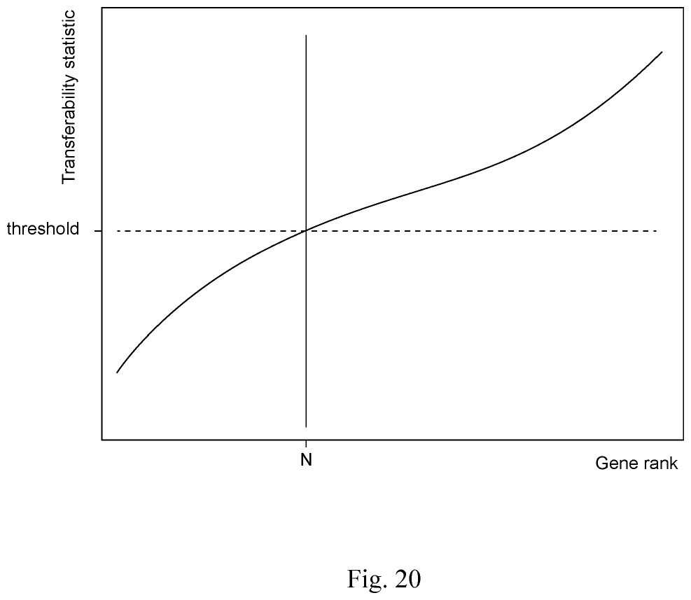

Referring to FIG. 19, a plot is provided of a K-S statistic versus gene rank. We computed the K-S statistics for 160 signature genes (98 genes from above and 62 genes from a separate signature). When sorted by rank, one can observe an initial increase in the K-S statistic slope at rank 136. Thus, the remaining 26 genes may be deemed “non-transferable” and removed from the model. FIG. 20 similarly shows a threshold in transferability statistic (e.g., located at an inflection point).

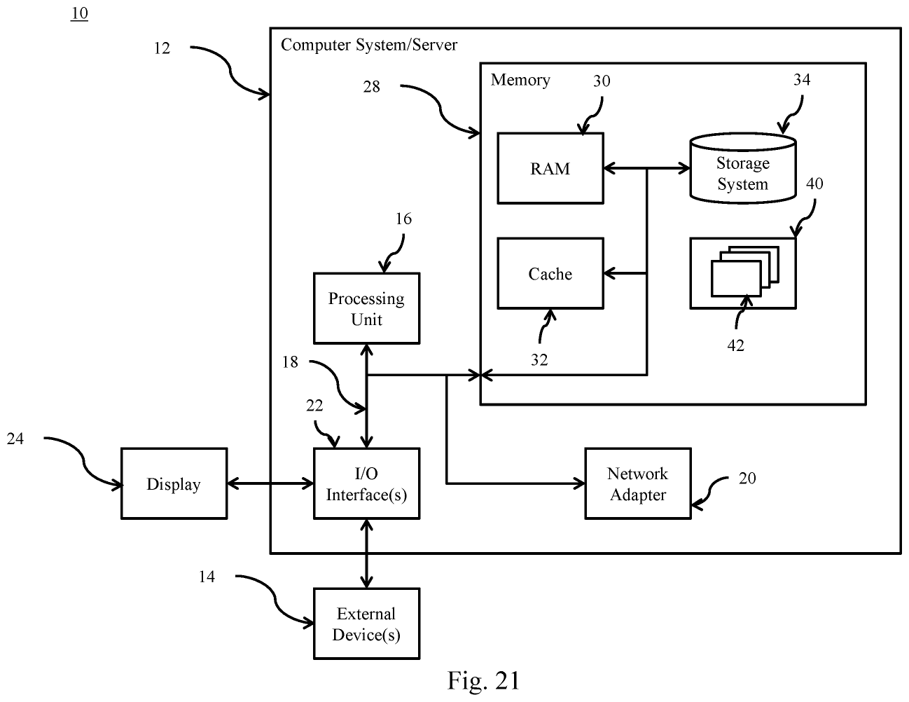

Referring now to FIG. 21, a schematic of an example of a computing node is shown. Computing node 10 is only one example of a suitable computing node and is not intended to suggest any limitation as to the scope of use or functionality of embodiments described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 21, computer system/server 12 in computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, Peripheral Component Interconnect Express (PCIe), and Advanced Microcontroller Bus Architecture (AMBA).

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the disclosure.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

The present disclosure may be embodied as a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. A method comprising:
reading a first signature, the first signature relating a first plurality of molecular biomarkers to a first of a plurality of output classifications;
for each of a plurality of datasets, normalizing an expression value of each of the first plurality of molecular biomarkers for each of the plurality of output classifications, yielding a plurality of normalized expressions, each associated with one of the first plurality of molecular biomarkers, one of the plurality of output classifications, and one of the plurality of datasets;
for each of the first plurality of molecular biomarkers, performing a pairwise comparison between the normalized expressions associated with that molecular biomarker, each pairwise comparison being between normalized expressions associated with a same output classification and a different dataset, thereby determining a transferability score for each of the plurality of molecular biomarkers;
ranking the first plurality of molecular biomarkers based on each's transferability score;
generating a second plurality of molecular biomarkers from the first plurality of molecular biomarkers by applying a transferability score threshold to the first plurality of molecular biomarkers; and
providing a transferrable signature, the transferrable signature relating the second plurality of molecular biomarkers to the first of the plurality of output classifications.

2. The method of claim 1, wherein each of the first plurality of molecular biomarkers is a gene.

3. The method of claim 1, wherein each of the first plurality of molecular biomarkers is a protein.

4. The method of claim 1, wherein each signature comprises a mapping function.

5. The method of claim 1, wherein each signature comprises a plurality of synaptic weights.

6. The method of claim 1, wherein each output classification comprises a phenotype.

7. The method of claim 6, wherein the phenotype is a disease phenotype.

8. The method of claim 1, wherein said normalizing comprises quantile normalization.

9. The method of claim 1, wherein said normalizing is to a predetermined reference distribution.

10. The methods of claim 1, wherein performing the pairwise comparison comprises computing a Kolmogorov-Smirnov statistic.

11. The method of claim 1, wherein determining the transferability score comprises computing a mean of the pairwise comparisons.

12. The method of claim 1, wherein the plurality of datasets comprises at least one dataset derived from each of a plurality of platform technologies.

13. The method of claim 12, wherein the platform technologies comprise microarrays and RNA-sequencing.

14. The method of claim 12, wherein the platform technologies comprise mass spectrometry, ELISA, antibody arrays, peptide fingerprinting, and/or protein barcoding.

15. The method of claim 12, wherein each of the plurality of datasets are derived from the same biological samples.

16-30. (canceled)

31. A computer program product for determining a transferable molecular biomarker signature, the computer program product comprising a computer readable storage medium having program instructions embodied therewith, the program instructions executable by a processor to cause the processor to perform a method comprising:
reading a first signature, the first signature relating a first plurality of molecular biomarkers to a first of a plurality of output classifications;
for each of a plurality of datasets, normalizing an expression value of each of the first plurality of molecular biomarkers for each of the plurality of output classifications, yielding a plurality of normalized expressions, each associated with one of the first plurality of molecular biomarkers, one of the plurality of output classifications, and one of the plurality of datasets;
for each of the first plurality of molecular biomarkers, performing a pairwise comparison between the normalized expressions associated with that molecular biomarker, each pairwise comparison being between normalized expressions associated with a same output classification and a different dataset, thereby determining a transferability score for each of the plurality of molecular biomarkers;
ranking the first plurality of molecular biomarkers based on each's transferability score;
generating a second plurality of molecular biomarkers from the first plurality of molecular biomarkers by applying a transferability score threshold to the first plurality of molecular biomarkers; and
providing a transferrable signature, the transferrable signature relating the second plurality of molecular biomarkers to the first of the plurality of output classifications.

32-44. (canceled)

45. A method comprising:
reading a first signature, the first signature relating a first plurality of molecular biomarkers to a first of a plurality of output classifications;
for each of a pair of datasets, each of the pair of datasets being derived from different platform technologies and each of the pair of datasets being derived from the same biological samples, determining a correlation coefficient for each of the first plurality of molecular biomarkers between the pair of datasets;
for each of the plurality of output classifications, determining a classification-specific correlation coefficient for each of the first plurality of molecular biomarkers between the pair of datasets;
ranking the first plurality of molecular biomarkers based on each's correlation coefficient and classification-specific correlation coefficient;
generating a second plurality of molecular biomarkers from the first plurality of molecular biomarkers by applying a rank threshold to the first plurality of molecular biomarkers; and
providing a transferrable signature, the transferrable signature relating the second plurality of molecular biomarkers to the first of the plurality of output classifications.

46-71. (canceled)

72. The method of claim 1, further comprising:
determining a second transferrable signature by:
reading the first signature;
for each of a pair of datasets, each of the pair of datasets being derived from different platform technologies and each of the pair of datasets being derived from the same biological samples, determining a correlation coefficient for each of the first plurality of molecular biomarkers between the pair of datasets;
for each of the plurality of output classifications, determining a classification-specific correlation coefficient for each of the first plurality of molecular biomarkers between the pair of datasets;
ranking the first plurality of molecular biomarkers based on each's correlation coefficient and classification-specific correlation coefficient;
generating a third plurality of molecular biomarkers from the first plurality of molecular biomarkers by applying a rank threshold to the first plurality of molecular biomarkers; and
providing a second transferrable signature, the transferrable signature relating the third plurality of molecular biomarkers to the first of the plurality of output classifications;
determining a third transferrable signature by determining an intersection or a union of the first and second transferable signatures.

73. (canceled)

74. The method of claim 1, further comprising:
determining a second transferable signature from the first transferrable signature by:
for each of a pair of datasets, each of the pair of datasets being derived from different platform technologies and each of the pair of datasets being derived from the same biological samples, determining a correlation coefficient for each of the second plurality of molecular biomarkers between the pair of datasets;
for each of the plurality of output classifications, determining a classification-specific correlation coefficient for each of the second plurality of molecular biomarkers between the pair of datasets;
ranking the second plurality of molecular biomarkers based on each's correlation coefficient and classification-specific correlation coefficient;
generating a third plurality of molecular biomarkers from the second plurality of molecular biomarkers by applying a rank threshold to the second plurality of molecular biomarkers; and
providing a second transferrable signature, the second transferrable signature relating the third plurality of molecular biomarkers to the first of the plurality of output classifications.

75. The method of claim 45, further comprising:
determining a second transferable signature from the first transferrable signature by:
for each of a plurality of datasets, normalizing an expression value of each of the second plurality of molecular biomarkers for each of the plurality of output classifications, yielding a plurality of normalized expressions, each associated with one of the second plurality of molecular biomarkers, one of the plurality of output classifications, and one of the plurality of datasets;
for each of the second plurality of molecular biomarkers, performing a pairwise comparison between the normalized expressions associated with that molecular biomarker, each pairwise comparison being between normalized expressions associated with a same output classification and a different dataset, thereby determining a transferability score for each of the second plurality of molecular biomarkers;
ranking the second plurality of molecular biomarkers based on each's transferability score;
generating a third plurality of molecular biomarkers from the second plurality of molecular biomarkers by applying a transferability score threshold to the second plurality of molecular biomarkers; and
providing a second transferrable signature, the second transferrable signature relating the third plurality of molecular biomarkers to the first of the plurality of output classifications.
